(19) United States
(12) Patent Application Publication
(10) Pub. No.: US 2023/0270899 A1
(43) Pub. Date: Aug. 31, 2023

(54) SANITISING APPARATUS AND METHOD

(71) Applicant: NOX WORLDWIDE LIMITED, Leeds (GB)

(72) Inventors: James Donaldson, Leeds (GB); Claire Anne, Leeds (GB)

(21) Appl. No.: 18/006,620

(22) PCT Filed: Jul. 28, 2021
(86) PCT No.: PCT/GB2021/051943

§ 371 Date: Jan. 24, 2023

(30) Foreign Application Priority Data
Jul. 29, 2020 (GB) 2011810.5
Nov. 20, 2020 (GB) 2018313.3

(51) Int. Cl. A61L 2/14; A61L 2/00; A61L 2/20; A61L 9/12

(52) U.S. Cl. CPC A61L 2/14; A61L 2/0094; A61L 2/202; A61L 9/122; A61L 2202/25; A61L 2202/26; A61L 2202/16; A61L 2202/11; A61L 2209/212; A61L 2202/122

(57) ABSTRACT

An apparatus for sanitising a target comprises a supply of a sanitising substance comprising a gas and/or plasma of nitric oxide and/or ozone, and a conduit arranged to direct the sanitising substance onto a target to sanitise the target. FIG. 13(a) shows a sanitiser product generator device 1200 which is arranged to release a sanitising product into an elevator 1220 via channel 1240. The sanitising product will destroy bacteria and virus within a mixing chamber before being released back into the lift shaft and lift cabin free from contaminants and may permeate the entire enclosed volume of the elevator, thereby destroying bacteria and virus in the air and on the surfaces of the elevator, circulating clean air and cleaning any contaminated air incoming via opening of the elevator doors or from passengers coughing and sneezing, for example.

The present invention relates to apparatus and methods of sanitising a target, and is concerned particularly, although not exclusively, with apparatus and methods for sanitising articles and spaces.

Previously considered approaches to sanitisation, such as the destruction of bacteria and viruses and other pathogens, include the use of liquid disinfectant and antibacterial agents, typically by spraying or rinsing, dependent upon the application. However, liquids are not always able to permeate efficiently the intended target, which may comprise an article or a physical space.

In another previously considered approach, radiation, such as ultraviolet radiation, is directed onto a target to destroy potentially harmful bacteria and viruses. However, this method also lacks complete effectiveness, particularly where the shape of the target is complex or convolute, as parts of an intended target surface may lie in a radiation shadow. The use of UV radiation is also problematic in some circumstances because of its potential to cause harm to human or animal life.

There is an increasing need to provide safe and efficient techniques for sanitising articles, surfaces and spaces across a wide range of industrial, commercial and domestic settings. Accordingly, embodiments of the present invention aim to provide apparatus and methods of safely and effectively sanitising a target, such as an article, surface or space, without the drawbacks seen in the prior art.

The present invention is defined in the attached independent claims, to which reference should now be made. Further, preferred features may be found in the sub-claims appended thereto.

According to one aspect of the present invention, there is provided an apparatus for sanitising a target, the apparatus comprising a supply of a sanitising substance comprising a gas and/or plasma of nitric oxide and/or ozone, wherein the apparatus is arranged to direct the sanitising substance onto a target to sanitise the target.

The apparatus may be arranged to direct the sanitising substance onto a target via a conduit and/or via a propelling device, such as, but not limited to, a fan.

The supply of sanitising substance may comprise a generator of gas or plasma. In a preferred arrangement, the supply comprises a generator of a cold plasma of nitric oxide and/or ozone.

The target may comprise a space, such as within a room, a elevator, conveyor, a vehicle or a container of articles. In a preferred arrangement, the target may comprise an enclosed space.

Alternatively, or additionally, the target may comprise a surface, such as a surface, more preferably an internal surface, of a vehicle, or a building structure, and/or an item of furniture, equipment or clothing, or packaging, an elevator or conveyor.

The target may comprise a human or an animal, or a part thereof, such as a limb. The apparatus may comprise a closure, for example a circumferential enclosure, or envelope, arranged to substantially enclose at least a part of a human or an animal.

The target may comprise an article, such as a tool, implement, utensil or the like.

The target may comprise a process, such as a manufacturing process or a packaging process.

The apparatus may comprise a mixing chamber for mixing the sanitising substance with air, wherein the apparatus comprises a filter, arranged in use to substantially remove the sanitising substance before the air is released from the chamber.

According to another aspect of the present invention, there is provided a method of sanitising a target, the method comprising directing a supply of sanitising substance comprising a gas or plasma of nitric oxide and/or ozone onto a target to sanitise the target.

The target may be according to any statement herein.

The method may include drawing air into a mixing chamber, mixing the air with the sanitising substance and releasing the air from the mixing chamber through a filter that is arranged to substantially remove the sanitising substance from the air.

The invention may include any combination of the features or limitations referred to herein, except such a combination of features as are mutually exclusive, or mutually inconsistent.

A preferred embodiment of the present invention will now be described, by way of example only, with reference to the accompanying diagrammatic drawings, in which:

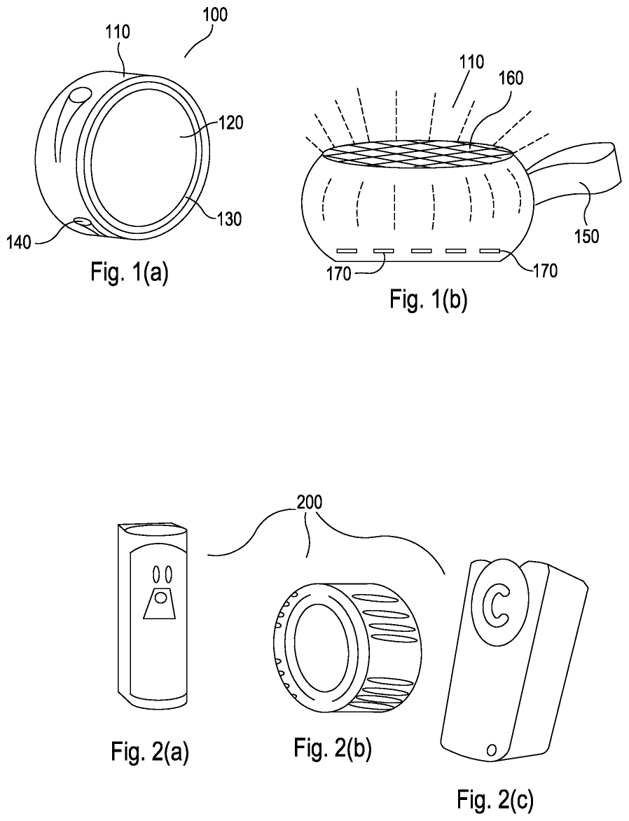

FIGS. 1(a) and 1(b) show schematically a dispensing device incorporating sanitising apparatus according to an embodiment of the present invention;

FIGS. 2(a) to 2(c) show schematically an alternative dispensing device incorporating sanitising apparatus in accordance with an embodiment of the present invention;

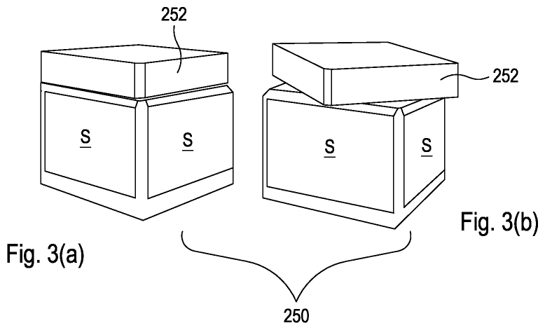

FIGS. 3(a) and 3(b) show schematically a further dispensing device incorporating sanitising apparatus in accordance with an embodiment of the present invention;

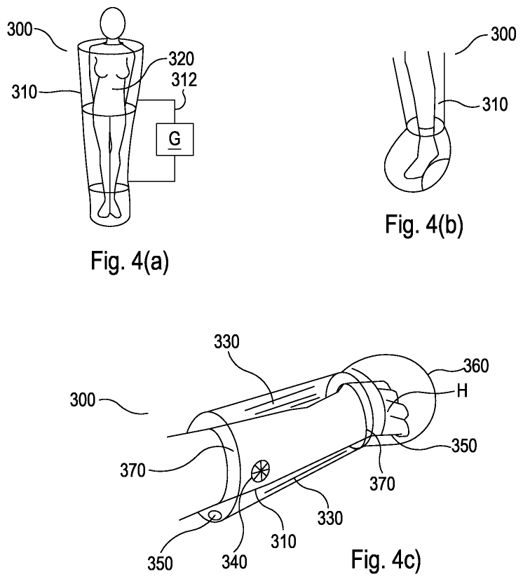

FIGS. 4(a) to 4(c) show schematically examples of enclosure devices for treating human patients, incorporating sanitising apparatus in accordance with an embodiment of the present invention;

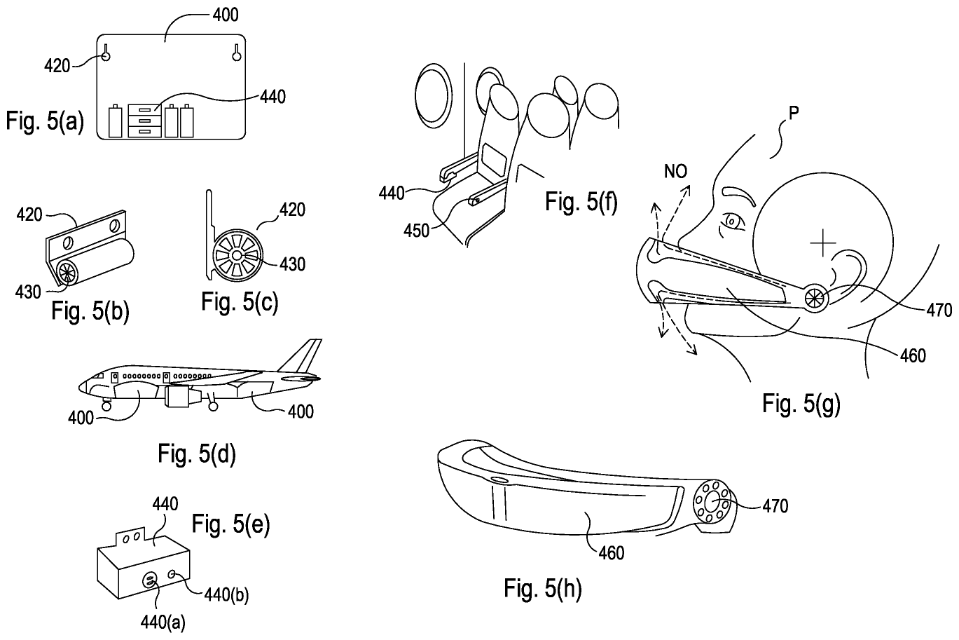

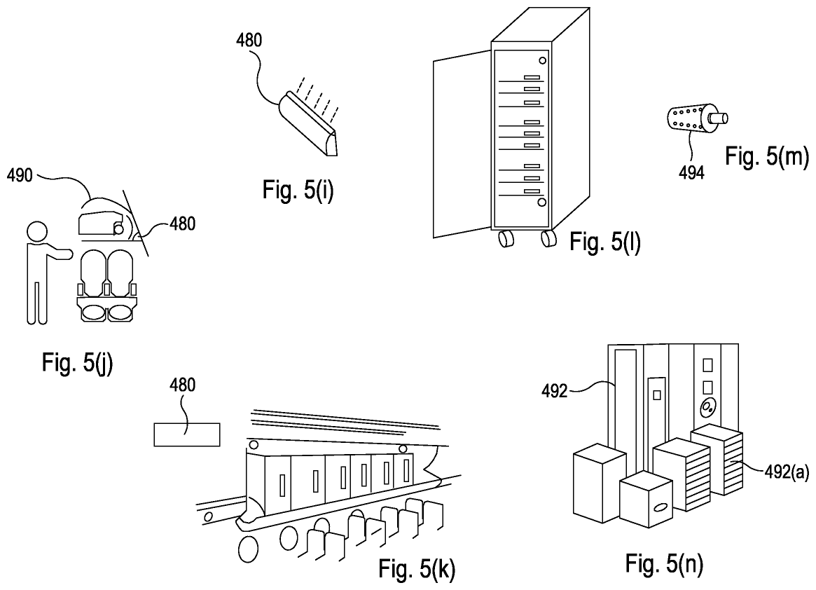

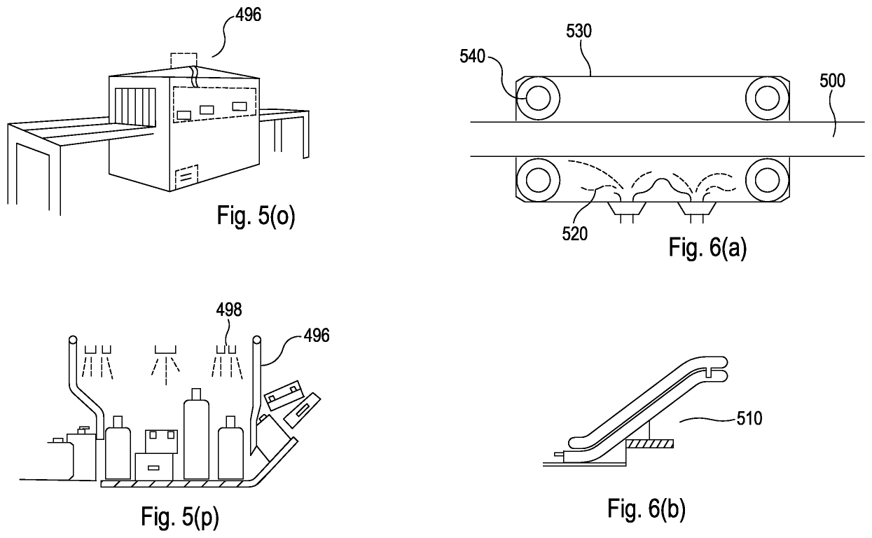

FIGS. 5(a) to 5(p) show schematically various apparatus and environments associated with travel and transportation incorporating sanitising apparatus in accordance with an embodiment of the present invention;

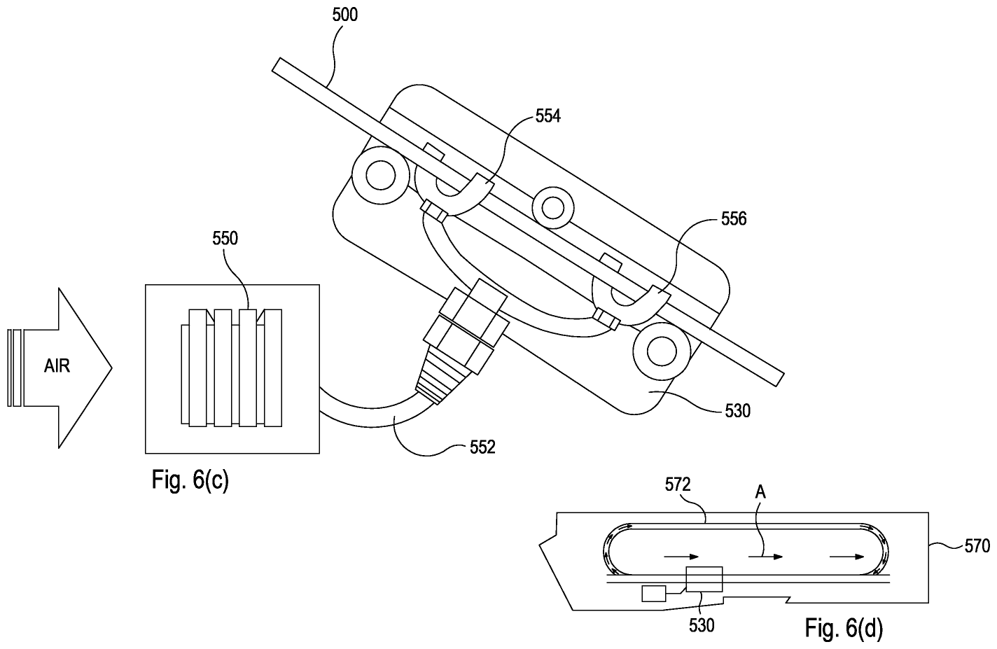

FIGS. 6(a) to 6(d) show schematically parts of an escalator incorporating sanitising apparatus in accordance with an embodiment of the present invention;

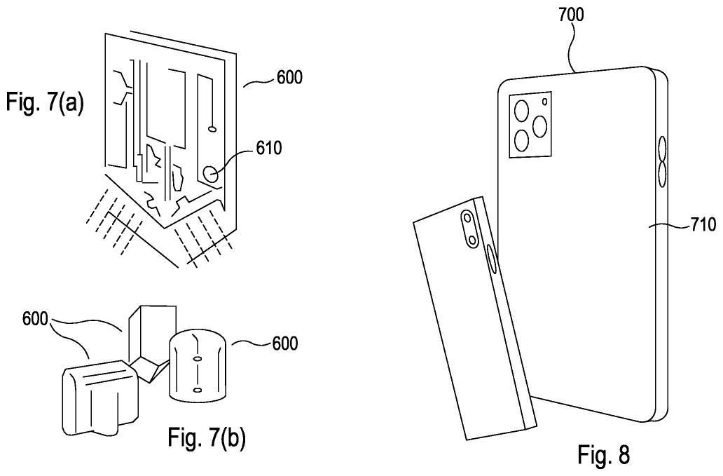

FIGS. 7(a) and 7(b) show schematically examples of hand driers incorporating sanitising apparatus in accordance with an embodiment of the present invention;

FIG. 8 shows schematically a cell phone case incorporating sanitising apparatus in accordance with an embodiment of the present invention;

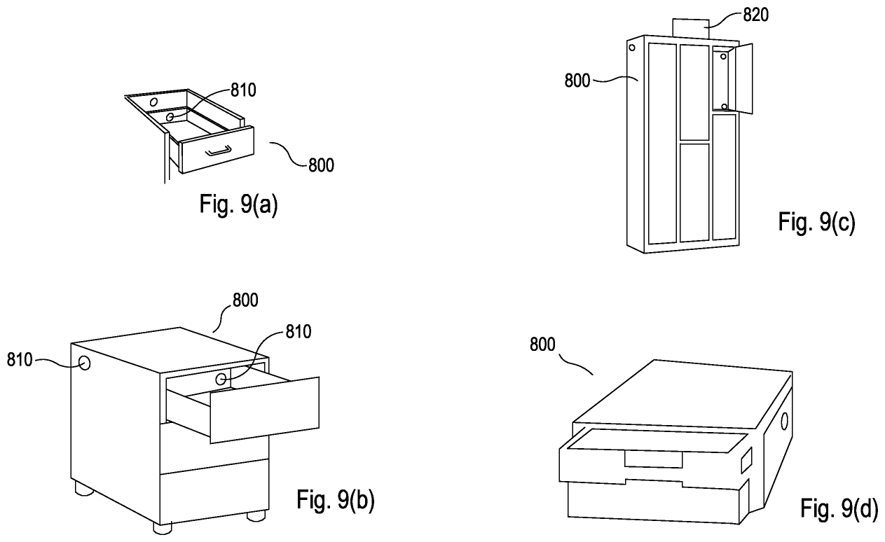

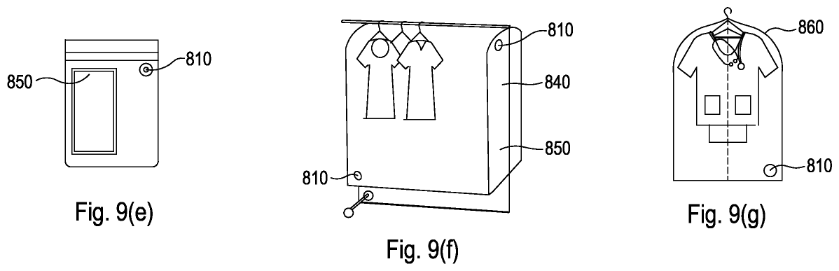

FIGS. 9(a) to 9(g) show schematically examples of storage apparatus incorporating sanitising apparatus in accordance with an embodiment of the present invention;

FIGS. 10(a) and 10(b) show schematically portable carrying apparatus incorporating sanitising apparatus in accordance with an embodiment of the present invention;

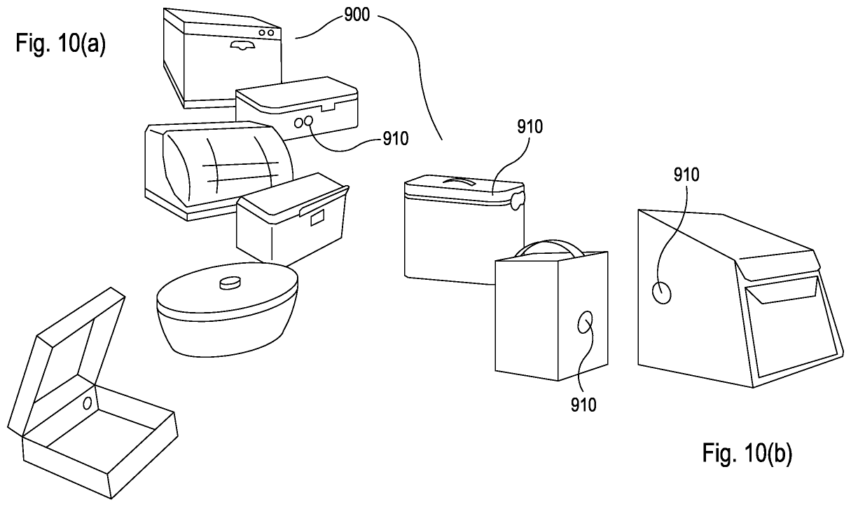

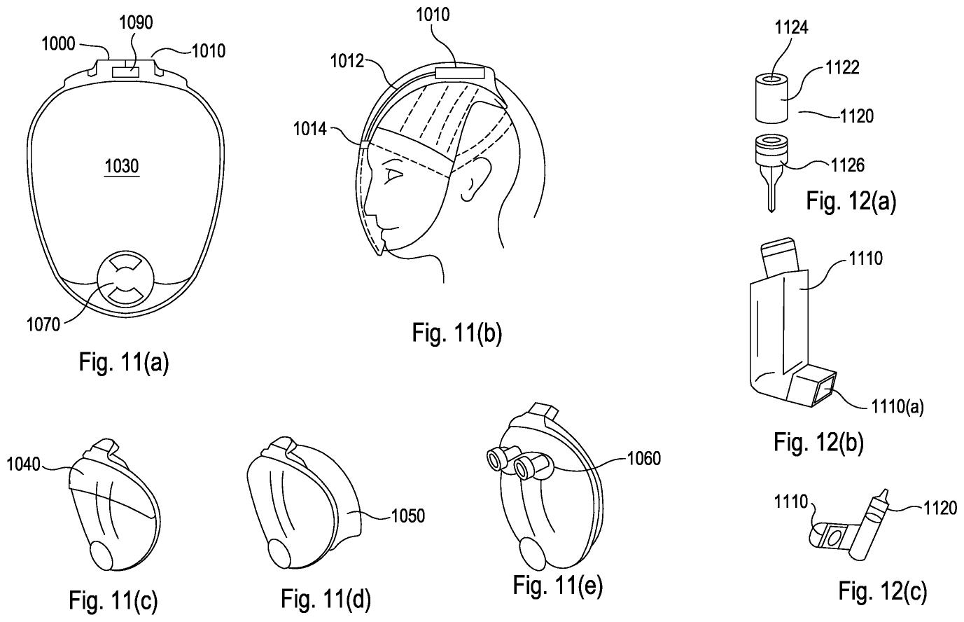

FIGS. 11(a) to 11(e) show schematically face masks incorporating sanitising apparatus in accordance with an embodiment of the present invention;

FIGS. 12(a) to 12(c) show an inhaler incorporating sanitising apparatus in accordance with an embodiment of the present invention;

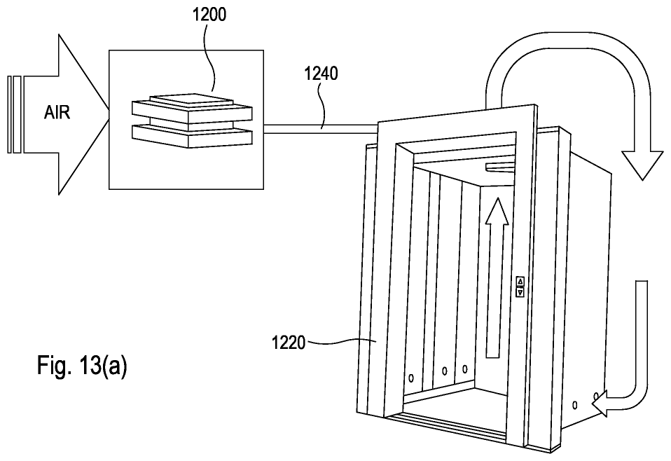

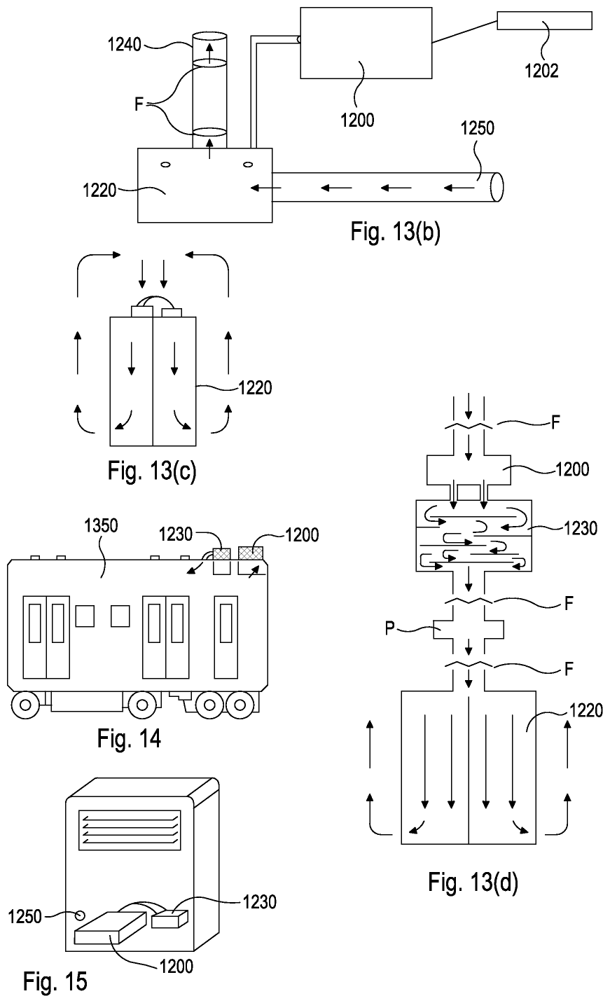

FIGS. 13(a) to 13(d) show schematically an elevator incorporating a sanitising apparatus to decontaminate the air, for example, in accordance with an embodiment of the present invention;

FIG. 14 shows schematically a vehicle incorporating a sanitising apparatus similar to that shown in FIGS. 13(a) to 13(d); and

FIG. 15 shows schematically a stand-alone air conditioning apparatus in accordance with an embodiment of the present invention.

Embodiments of the present invention employ miniaturised generators of gases and/or plasmas such as of nitric oxide and/or ozone to sanitise/sterilise/disinfect/render aseptic articles, surfaces and/or spaces. In some embodiments the sanitising substance is directed onto a target, which may comprise an article, surface or a space, for example, via a conduit and/or via a propelling device, such as, but not limited to, a fan.

Examples of techniques for generating plasmas and gases which may be suitable for adaptation for use with the present invention may be found, for example, in UK Patents GB 2532195 B, GB2548382 B and GB 2548557 B, the entire contents of which are hereby incorporated by reference.

The term “product” will be used below to refer to a sanitising substance, such as a plasma or gas of nitric oxide and/or ozone. The nature and composition of the product will be chosen in each case depending on the application of the gas/plasma, and the nature of the target (air/spaces, surfaces etc). In some cases, the product will comprise substantially only NO and, in some cases, substantially only ozone. In some applications there will be a combination of NO and ozone, with the respective concentrations determined by the specific use. Where skin treatment is involved, only NO will be used.

In some embodiments, the product is used to sanitise the air, so that air is mixed with the product. The sanitising of the air may take place in a mixing chamber and the product may be filtered out from the air before the air is released from the mixing chamber. This is particularly the case where the product may be considered harmful to humans.

In several embodiments, a miniature combined inlet, fan and product generator is provided to draw air in and expose the air to sanitising product.

Turning now to the drawings, FIG. 1(a) shows generally at 100 a simple apparatus for sanitising an environment. In this example, the apparatus may be considered as a personal, or portable, apparatus. The device comprises a housing 110, a push-button 120, an illuminated bezel 130 and release vents 140. The device may be powered by a battery (not shown) and may be mounted in, or on, a confined space, such as a drawer, cupboard or container. To activate the device a user presses the button 120 whereupon a generator (not shown) located within the housing releases product into the vicinity. FIG. 1(b) shows a variant for personal use which may be smaller and may be conveniently carried in a handbag or similar. A carry handle 150 and a mesh outlet vent 160 are provided, as is an activation switch (not shown) which may be configured to activate the release of product either on a timed basis, or else on sensing movement for example. Air is drawn into the device via inlet vents 170. A generator (not shown) within the device produces product, to which the air is exposed prior to the air being released. The device is particularly suited to a short duration of operation.

FIGS. 2 (a) to 2(c) show generally at 200 examples of a room sanitiser/freshener which may be freestanding or else may be mounted on a wall (not shown), for example of a public restroom, and which may be mains powered or battery powered. The devices 200 may be arranged to dispense product based on a timer or upon sensing of movement and may also produce an odour-neutralising effect in the room.

FIGS. 3(a) and 3(b) show generally at 250 a room sanitiser which may be freestanding and which may be mains powered or battery powered. 3(a) shows the sanitiser in a non-functional arrangement, with bezel 252 flush with the sides (S) of the sanitiser. 3(b) shows the sanitiser in a functional arrangement, with bezel 252 rotated about a vertical plane with respect to the sides of the sanitiser. The apparatus may be considered for domestic, personal or portable use and may be activated in small to medium enclosures to sanitise personal or household belongings. Swivelling the bezel 252 may be arranged to activate the device.

FIGS. 4(a) to 4(c) show, generally at 300, examples of enclosures for use in treating humans or animals with product. In each case an envelope, or sleeve, 310 surrounds a treatment site and product is generated and introduced into the envelope. FIG. 4(a) is an example of a full body sleeve which encloses the body of a patient 320 up to the head. In this example, product is generated at an external generator G and introduced to the envelope via tubing 312. In FIG. 4(b) it is the foot of a patient that is enclosed by the sleeve and in FIG. 4(c) the arm of a patient is enclosed. In each case the sleeve is inflatable and transparent, allowing the treatment site to be visible to a clinician, thereby removing the need for unnecessary changing of any dressings. Ribs and tubes 330 help to provide structure and air is introduced into the sleeve at a miniature fan/inlet/product generator 340, which may be push-activated. The miniature generator causes the air to be sanitised as it enters, whilst air exits via outlets 350. The enclosure has a one way valve (not shown) for delivering the product to the treatment site, and a sensor (not shown) to ensure that the product levels are monitored for optimum sterilisation and wound healing.

In FIG. 4(c), the patient's hand H can be seen within a sealed enclosure 360, allowing free movement of the fingers. The treatment site is sealed at either end by seals 370.

The treatment enclosures may comprise modular enclosures that may be combined. Larger enclosures, such as that shown in FIG. 4 (a), may be used to treat a patient in a clinic, whereas smaller enclosures, such as are shown in FIGS. 4(b) and 4(c) may be used by the patient at home.

FIGS. 5(a) to 5(p) show examples of apparatus used to provide safe sanitation in a transport environment, more particularly in the field of air travel in this example.

FIG. 5(a) shows generally at 400 a hold storage space of an aircraft. The hold contains baggage 410 which may be sanitised using apparatus 420 in accordance with the present invention which is shown in more detail in FIGS. 5(b) (perspective) and 5(c) (end view). The apparatus 420 comprises a generator of product which may be fixed to the interior of the baggage hold space. Electrically operated rotors 430 cause product to circulate steadily within the hold during the flight so as to sanitise the luggage.

FIGS. 5(e) and 5(f) show a user terminal 440 which may be mounted to a seat arm 450 of the aircraft. The terminal comprises an outlet 440(a) for releasing sanitising product into the cabin. This may be used for example when the cabin is being cleaned and prepared for passengers or may be used in-flight. Beside the outlet 440(a) is a plug-in socket 440(b) for personal use by a passenger P, as shown in FIG. 5(g). The passenger P wears a personal sanitising mask device 460 which is supported by the ears, in the manner of conventional headphones. The mask device 460 delivers sanitising product from the outlet 440(a) to the front of the passenger's face around the mouth and nose area. As an alternative, clean air may be drawn into the device 460 and a miniature inlet/fan/generator 470 on the device 460 may introduce product into the air stream. The sanitised air flows out of the device 460 to create a shield of NO and clean air, for example, labelled NO.

FIG. 5(i) shows a small generator 480 of sanitising product for locating in or beside a cabin locker 490 (FIGS. 4(j) and 4(k)) of the aircraft. The generator 480 may be arranged to introduce product into the locker 490 continuously when loaded or during a timed operation. For example, the closure of the locker door may be arranged to trigger the release of product into the locker.

FIGS. 5(l)-5(o) show catering lockers 492 for the aircraft. These store food items for the flight and the interior can be sanitised using a small product generator 494 which be operated when the door is closed. For example, food trays 492 (a) may be sanitised when they are first loaded into the lockers and then, after use, the trays may be sanitised again after stowing.

FIG. 5(o) shows generally at 496 a luggage scan device as used in an airport. The luggage scan device 496 can also be used to sanitise the luggage at the same time, as shown schematically in FIG. 5(p) by incorporating product generators 498 within the device 496. One or more of these can be retrofitted to existing apparatus and incorporated into an enclosed, or semi-enclosed belt and roller conveyor assembly, without slowing the transfer of luggage. The apparatus can be adapted to work with existing belt lines or into belt lines for security checks, for example when using X-ray or other scanner machinery, such as in baggage collection conveyors and security bag scanners. As an alternative to the internally mounted product generator 498, an external generator may be provided which may be connected to the enclosure 496 by tubing.

FIG. 6(a) shows a portion of a handrail 500 which is part of an escalator system 510 shown in FIG. 6(b). The handrail 500 travels in a continuous loop and during its return, within the enclosed structure of the escalator, the handrail 500 may be sanitised using product that is generated by a generator 520. A sanitiser body 530 may enclose a portion of the handrail with rubber containment rollers 540 while it is being sanitised. FIG. 6(c) shows an alternative embodiment in which a product generator 550 directs sanitising product onto and around a handrail 500 in a sanitiser body/dwell enclosure 530 through a channel 552, and out of apertures on portions 554,556.

The rails pass through an enclosed body in which they are exposed to the product, typically ozone, which fills the enclosed space. The appropriate concentration of product and the optimum residence/exposure time of the handrail in the enclosure 530 are calculated and may be monitored by sensors (not shown). End seals prevent the ozone from leaking out of the dwell enclosure 530.

Although the example of an escalator has been described, the apparatus may also be used on handrails and other parts of e.g. travelators, moving walkways and baggage conveyors.

FIG. 6(d) shows schematically a travelator/moving walkway, generally at 570. A moving platform 572 conveys passengers in the direction of arrows A. When the platform returns, it passes through a sealed enclosure 530 connected to a generator 550 which ensures that the platform 572 is exposed to ozone or NO for sanitisation.

FIG. 7(a) shows schematically at 600 a hand drier incorporating a generator 610 which is arranged to introduce sanitising product into the air stream that is then directed to the hands (not shown) of a user. FIG. 7(b) shows alternative examples of hand drier that may incorporate the device. These examples may find application, for example, in public restrooms.

FIG. 8 shows generally at 700 a sanitising device for use with personal equipment, which in the example is a cell phone 710. The device comprises a case for the cell phone which has a miniature product generator built in, and which derives electrical power from the phone, for example by induction. The device 700 may release product into the ambient air or may supply it through a tube (not shown) to a face mask (not shown).

FIGS. 9(a) to 9(g) show examples of storage units, cupboards, drawers, cabinets and lockers generally at 800 which may utilise the product to sanitise items stored within them, such as equipment, utensils, apparatus or clothing, for example the clothing, footwear and personal belongings of people, such as medical workers for example (none shown). Miniature generators 810, located within the (optionally sealed) units 800, may release sanitising product automatically, for example upon closure, or else may do so manually upon user-initiated operation, such as by pressing a button of the device. The product, optionally ozone, may be delivered at a pre-calculated, monitored dose over a predetermined duration, to sterilise and make safe the contents without causing damage, and the ozone may be filtered before release from the enclosure. Exposure to sanitising product in this way may also remove unwanted odours, for example caused by bacteria, as a result. The apparatus 100 described with reference to FIG. 1, may be used for this purpose. Alternatively, an RF or other communications link (not shown) may be provided to control the operation of the product generator.

The miniature generators 810 may comprise integrated inlet/fan/generator units that draw in air and sanitise it with generated product. Alternatively, in the example of FIG. 9(c), a larger unit may be provided with product from an externally sited generator 820, which may supply several cabinets and may provide for intermittent release of product, such as NO or ozone.

FIG. 9 (e) shows a fold-over sanitizing bag 830, for personal belongings, featuring a mini generator 810 and an air outlet (not shown).

FIG. 9(f) shows a clothes rack 840 incorporating an enclosure 850, which is in this example transparent, provided with miniatured inlet/fan/generators 810 and air outlets (not shown). The clothes rack 840 is suited to use in retail premises where clothes must be sanitised after being tried-on (but not purchased) by customers, before being returned to display.

FIG. 9(g) shows a personal clothes store/carrier 860 incorporating a mini fan/inlet/generator 810. This may be used, for example, to sanitise work clothes or belongings or wearable equipment.

The apparatus may also be used in the storage of food items. Currently many people spend considerable time disinfecting fresh food and food packaging when bringing it into their homes. Viruses and bacteria can survive at low temperatures such as are commonly found in refrigerators. A drawer or box in accordance with embodiments of the present invention can be used to disinfect not only personal items and deliveries of goods, but also food. Indeed, fresh food, including fruit and vegetables, may be kept fresh for longer when exposed to sanitising product in this way.

FIGS. 10(a) and 10(b) show examples of storage containers, generally at 900, which include sanitising product generation in accordance with the present invention. The examples in FIG. 10(b) include hard and soft cool boxes for the transportation of food or for use in medical applications, and/or for personal belongings storage and transport for e.g. handbags, and/or for holding and disinfecting particularly renowned bacteria-attracting devices and articles. Miniature product generators 910 may be incorporate into the side walls, bases or lids of the containers.

FIGS. 11(a) to 11 (e) show examples of face masks, generally at 1000. The masks 1000 include a generator 1010 which introduces sanitising product into the air drawn into the mask by the action of the wearer breathing. The generators may conveniently be powered by a battery pack (not shown) and the masks include a visor portion 1030 for covering the face. Variants include a hinged visor 1040 (FIG. 11(c)), a solid hinged hair net 1050 (FIG. 11(d)) and an interchangeable loupe type visor 1060 (FIG. 11(e)). In each case a HEPA waste filter 1070 may be provided (FIG. 11 (a)) as may a gas catalyst/filter (not shown) and a battery level indicator 1090 (FIG. 11(a)).

In FIG. 11 (b), air is drawn into a top portion of the mask by generator 1010 which then causes the drawn in air to mix with product in mixing chamber 1012. The sanitised air passes through a filter 1014, to remove product, before reaching the face of the wearer.

Any filter can be permeated by the product, so that viruses and bacteria caught by the filter can be destroyed. Without this, filters could become overloaded, and ultimately overwhelmed.

FIGS. 12(a) to 12(c) together show an inhaler device 1100 comprising a body 1110 and a replaceable cartridge 1120 which is arranged to release sanitising product through a mouthpiece 1110(a) of the body 1100 in use. The cartridge 1120 comprises a battery power unit 1122, having an actuation button 1124, and a generator unit 1126 for generating the product.

FIG. 13(a) shows a sanitiser product generator device 1200 which is arranged to release a sanitising product into an elevator 1220 via channel 1240. The sanitising product will destroy bacteria and virus within a mixing chamber before being released back into the lift shaft and lift cabin free from contaminants and may permeate the entire enclosed volume of the elevator, thereby destroying bacteria and virus in the air and on the surfaces of the elevator, circulating clean air and cleaning any contaminated air incoming via opening of the elevator doors or from passengers coughing and sneezing, for example. As discussed above, the degree of penetration is greater than liquid sprays. Furthermore, the application of product according to the embodiments of the present invention does not require the elevator to be temporarily suspended from service, as would be the case with liquid sprays and UV exposure.

FIG. 13(b) shows schematically, in more detail, apparatus for sanitising the air for the embodiment of FIG. 13(a). The generator 1200 is connected to a power supply 1202 and generates a sanitising plasma, preferably of ozone. The plasma travels to a mixing/dwell chamber 1230 where it mixes with air drawn in from the elevator car through conduit 1250. The sanitised air then passes through catalyst filters F before being reintroduced to the elevator car through conduit 1240.

FIG. 13(c) shows schematically the flow of air into the elevator car, out into the elevator shaft and then back to the car.

FIG. 13(d) shows schematically an alternative configuration in which the air is drawn through a filter F to the generator 1200 and into a dwell chamber 1230 by a pump P, through another filter F before entering the elevator car 1220.

The approach can be adapted for other vehicles. FIG. 14 shows schematically a rail car 1300. The vehicle could also be a tram/bus or ambulance, as non-limiting examples. A generator 1200 is located externally on the roof of the car 1300, but it could be located internally. A flow path that is similar to the above example of the elevator car is established, in which sanitising product is mixed with drawn-in air in a dwell chamber 1230 before the product is filtered out and the sanitised air is released into the car.

FIG. 15 shows schematically a stand-alone air conditioning unit with an inlet 1250 introducing ambient air to a product generator before passing through a dwell chamber 1230. Thereafter, the air is conditioned and released.

In accordance with the present invention, the generator can be of various sizes, depending upon the application and/or the intended purpose (e.g. disinfection, decontamination of a surface, material, object or volume of enclosed space).

For smaller applications, a combined inlet fan/generator, which may also comprise an on-off control, such as a push button or switch, is preferred. The units may be battery powered, or mains powered, or may derive power in some other way.

The targets can be grouped broadly into two categories. A first category is surfaces, or objects and a second category is spaces. Where there is a risk of a human inhaling the product, a filter may be employed to scrub the air of product prior to release into an atmosphere.

Whilst endeavouring in the foregoing specification to draw attention to those features of the invention believed to be of particular importance, it should be understood that the applicant claims protection in respect of any patentable feature or combination of features referred to herein, and/or shown in the drawings, whether or not particular emphasis has been placed thereon.

1. An apparatus for sanitising a target, the apparatus comprising a supply of a sanitising substance comprising a gas and/or plasma of nitric oxide and/or ozone, wherein the apparatus is arranged to direct the sanitising substance onto a target to sanitise the target.

2. An apparatus according to claim 1, wherein the supply of sanitising substance comprises a generator of gas or plasma.

3. An apparatus according to claim 1, wherein the supply comprises a generator of a cold plasma of nitric oxide and/or ozone.

4. An apparatus according to claim 1, wherein the target comprises a space, such as within a room, an elevator, conveyor, a vehicle or a container of articles.

5. An apparatus according to claim 1, wherein the target comprises a surface, such as a surface of a vehicle, or a building structure, and/or an item of furniture, equipment or clothing, or packaging, an elevator or conveyor.

6. Apparatus according to claim 1, wherein the target comprises a human or an animal, or a part thereof, such as a limb.

7. Apparatus according to claim 6, wherein the apparatus comprises a closure, for example a circumferential enclosure, or envelope, arranged to substantially enclose at least a part of a human or an animal.

8. Apparatus according to claim 1, wherein the target comprises an article, such as a tool, implement or utensil.

9. Apparatus according to claim 1, wherein the target comprises a process, such as a manufacturing process or a packaging process.

10. Apparatus according to claim 1, wherein the apparatus comprises a mixing chamber for mixing the sanitising substance with air, wherein the apparatus comprises a filter, arranged in use to substantially remove the sanitising substance before the air is released from the chamber.

11. A method of sanitising a target, the method comprising directing a supply of sanitising substance comprising a gas or plasma of nitric oxide and/or ozone onto a target to sanitise the target.

12. A method according to claim 11, wherein the method comprises a method of sanitising a target including any of the following: a space, such as within a room, an elevator, conveyor, a vehicle or a container of articles; a surface, such as a surface of a vehicle, or a building structure, and/or an item of furniture, equipment or clothing, or packaging; an elevator or conveyor; a human or an animal, or a part thereof, such as a limb; an article, such as a tool, implement or utensil.

13. A method according to claim 11, wherein the method includes drawing air into a mixing chamber, mixing the air with the sanitising substance and releasing the air from the mixing chamber through a filter that is arranged to substantially remove the sanitising substance from the air.
